(19) United States
(12) Patent Application Publication
(10) Pub. No.: US 2026/0341421 A1
(43) Pub. Date: Oct. 29, 2026

(54) IDENTIFICATION OF NEW CHEMICAL COMPOUNDS WITH DESIRED PROPERTIES

(71) Applicant: BAYER AKTIENGESELLSCHAFT, Leverkusen (DE)

(72) Inventors: Fuad Abdallah, Leverkusen (DE); Silva Galvez-Cerezo, Leverkusen (DE); Bing Ashley Liang O'Dowd, Leverkusen (DE); Anne Decor, Leverkusen (DE); Stephan Voelkening, Leverkusen (DE)

(21) Appl. No.: 19/107,190

(22) PCT Filed: Aug. 23, 2023
(86) PCT No.: PCT/EP2023/073095

§ 371 Date: Feb. 27, 2025

(30) Foreign Application Priority Data
Aug. 31, 2022 (EP) 22193037.3

(51) Int. Cl. G16C 20/30; G06N 3/0455; G16C 20/20; G16C 20/70

(52) U.S. Cl. CPC G16C 20/30; G06N 3/0455; G16C 20/20; G16C 20/70

(57) ABSTRACT

Systems, methods, and computer programs disclosed herein relate to identifying new chemical compounds having a desired property profile using a trained machine learning model.

### FIELD

Systems, methods, and computer programs disclosed herein relate to identifying new chemical compounds having a desired property profile using a trained machine learning model.

### BACKGROUND

In the research and development departments of the chemical industry, new chemical compounds are constantly being synthesized and their properties characterized in order to develop new drugs, crop protection products and/or other products with improved properties.

The search for new compounds with improved properties can proceed in several phases. In a first phase, a large number of existing compounds can be screened for one or more properties (e.g., a biological activity). Compounds that exhibit the one or more properties (e.g., the biological activity) can then be the starting point for an optimization as so-called lead compound. The chemical structure of a lead compound (lead structure) can serve as a starting point for chemical modifications to improve efficacy, selectivity, toxicity, safety, solubility, and/or other properties.

Ways to speed up the process are constantly being sought, as the production of chemical compounds and their characterization cost both time and money.

A. Tevosyan et al. disclose a method to improve chemical property prediction performance of machine learning models by incorporating additional information on correlated molecular descriptors in the representations learned by variational autoencoders (Improving VAE based molecular representations for compound property predictions, DOI: 10.1186/s13321-022-00648-x).

There are approaches to first generate new chemical compounds in the computer (in silico) and calculate their properties, and then chemically synthesize and test promising candidates.

For example, R. Gomez-Bombarelli et al. disclose a method for automatic chemical design using a deep neural network (Automatic Chemical Design Using a Data-Driven Continuous Representation of Molecules, ACS Cent. Sci. 2018, 4, 268-276). The deep neural network comprises three units: an encoder, a decoder, and a predictor. The encoder converts the discrete representation of a molecule into a real-valued continuous vector, and the decoder converts such a continuous vector back to a discrete molecular representation. The predictor estimates chemical properties from the latent continuous vector representation of the molecule. The predictor is a multi-layer perceptron and thus latent continuous vectors are mapped to the chemical properties via a non-linear function. Such a non-linear mapping between the latent space representation and chemical properties makes it difficult to identify, decode and optimize molecules with desired properties since they could be located in multiple locations in the latent space.

S. Mohammadi et al. therefore propose to use linear units for property prediction (Penalized Variational Autoencoder for Molecular Design, DOI: 10.26434/chemrxiv.7977131.v2). The linear prediction unit can be inverted in order to map back to the latent space starting with a property vector, without prior knowledge of a molecular structure. However, the prediction of new chemical compounds based solely on a desired property profile works only to a very limited extent in practice. On one hand side, such completely new chemical compounds must first be synthesized; chemical building blocks for generating the new chemical structures are often not available. On the other hand side, the predicted chemical compounds very often do not perform as hoped (predicted) in testing.

### SUMMARY

These and further problems are solved by the subject matter of the independent claims. Preferred embodiments can be found in the dependent claims, the present description, and the drawings.

Therefore, in a first aspect, the present disclosure provides a computer-implemented method, the method comprising:
providing a trained machine learning model, the trained machine learning model comprising an encoder, a decoder, and a linear transformation unit,
   wherein the encoder is configured and trained to convert a discrete molecular representation of a chemical compound into an embedding in continuous latent space,
   wherein the decoder is configured and trained to convert an embedding in the continuous latent space into a discrete molecular representation of a chemical compound,
   wherein the linear transformation unit is configured and trained to map an embedding in the continuous latent space to a property vector representing one or more properties of a chemical compound,
receiving a first molecular representation of a first lead compound and a second molecular representation of a second lead compound,
converting the first molecular representation of the first lead compound into a first embedding representing the first lead compound in the continuous latent space via the encoder,
converting the second molecular representation of the second lead compound into a second embedding representing the second lead compound in the continuous latent space via the encoder,
selecting a third embedding within the continuous latent space between the first embedding and the second embedding, the third embedding representing a candidate chemical compound in the continuous latent space,
converting the third embedding into a discrete molecular representation of the candidate chemical compound via the decoder,
outputting the discrete molecular representation of the candidate chemical compound and/or another representation of the candidate chemical compound.

In another aspect, the present disclosure provides a computer system comprising:
a processor; and
a memory storing an application program configured to perform, when executed by the processor, an operation, the operation comprising:
   providing a trained machine learning model, the trained machine learning model comprising an encoder, a decoder, and a linear transformation unit,
      wherein the encoder is configured and trained to convert a discrete molecular representation of a chemical compound into an embedding in continuous latent space,
      wherein the decoder is configured and trained to convert an embedding in the continuous latent space into a discrete molecular representation of a chemical compound,
      wherein the linear transformation unit is configured and trained to map an embedding in the continuous latent space to a property vector representing one or more properties of a chemical compound,
receiving a first molecular representation of a first lead compound and a second molecular representation of a second lead compound,
converting the first molecular representation of the first lead compound into a first embedding representing the first lead compound in the continuous latent space via the encoder,
converting the second molecular representation of the second lead compound into a second embedding representing the second lead compound in the continuous latent space via the encoder,
selecting a third embedding within the continuous latent space between the first embedding and the second embedding, the third embedding representing a candidate chemical compound in the continuous latent space,
converting the third embedding into a discrete molecular representation of the candidate chemical compound via the decoder,
outputting the discrete molecular representation of the candidate chemical compound and/or another representation of the candidate chemical compound.

In another aspect, the present disclosure provides a non-transitory computer readable storage medium having stored thereon software instructions that, when executed by a processor of a computer system, cause the computer system to execute the following steps:
providing a trained machine learning model, the trained machine learning model comprising an encoder, a decoder, and a linear transformation unit,
   wherein the encoder is configured and trained to convert a discrete molecular representation of a chemical compound into an embedding in continuous latent space,
   wherein the decoder is configured and trained to convert an embedding in the continuous latent space into a discrete molecular representation of a chemical compound,
   wherein the linear transformation unit is configured and trained to map an embedding in the continuous latent space to a property vector representing one or more properties of a chemical compound,
receiving a first molecular representation of a first lead compound and a second molecular representation of a second lead compound,
converting the first molecular representation of the first lead compound into a first embedding representing the first lead compound in the continuous latent space via the encoder,
converting the second molecular representation of the second lead compound into a second embedding representing the second lead compound in the continuous latent space via the encoder,
selecting a third embedding within the continuous latent space between the first embedding and the second embedding, the third embedding representing a candidate chemical compound in the continuous latent space,
converting the third embedding into a discrete molecular representation of the candidate chemical compound via the decoder,
outputting the discrete molecular representation of the candidate chemical compound and/or another representation of the candidate chemical compound.

### SHORT DESCRIPTION OF THE DRAWINGS

FIG. 1 shows schematically an example of a machine learning model of the present disclosure.

FIG. 2 shows schematically an example of training a machine learning model of the present disclosure.

FIG. 3 (a), (b) and (c) show schematically by way of example the use of the trained machine learning model for prediction.

FIG. 4 illustrates a computer system according to some example implementations of the present disclosure.

FIG. 5 shows an embodiment of the method for identifying a candidate chemical compound having a desired property profile in form of a flow chart.

### DETAILED DESCRIPTION

The invention will be more particularly elucidated below without distinguishing between the aspects of the disclosure (method, computer system, computer-readable storage medium). On the contrary, the following elucidations are intended to apply analogously to all the aspects of the disclosure, irrespective of in which context (method, computer system, computer-readable storage medium) they occur.

If steps are stated in an order in the present description or in the claims, this does not necessarily mean that the disclosure is restricted to the stated order. On the contrary, it is conceivable that the steps can also be executed in a different order or else in parallel to one another, unless one step builds upon another step, this absolutely requiring that the building step be executed subsequently (this being, however, clear in the individual case). The stated orders are thus preferred embodiments of the invention.

As used herein, the articles “a” and “an” are intended to include one or more items and may be used interchangeably with “one or more” and “at least one.” As used in the specification and the claims, the singular form of “a”, “an”, and “the” include plural referents, unless the context clearly dictates otherwise. Where only one item is intended, the term “one” or similar language is used. Also, as used herein, the terms “has”, “have”, “having”, or the like are intended to be open-ended terms. Further, the phrase “based on” is intended to mean “based at least partially on” unless explicitly stated otherwise. Further, the phrase “based on” may mean “in response to” and be indicative of a condition for automatically triggering a specified operation of an electronic device (e.g., a controller, a processor, a computing device, etc.) as appropriately referred to herein.

Some implementations of the present disclosure will be described more fully hereinafter with reference to the accompanying drawings, in which some, but not all implementations of the disclosure are shown. Indeed, various implementations of the disclosure may be embodied in many different forms and should not be construed as limited to the implementations set forth herein; rather, these example implementations are provided so that this disclosure will be thorough and complete, and will fully convey the scope of the disclosure to those skilled in the art.

The present disclosure provides means for predicting candidate chemical compounds with a desired property profile based on at least two lead compounds.

The term “chemical compound” is understood to mean a pure substance consisting of atoms of two or more chemical elements, where (in contrast to mixtures) the atomic species are in a fixed ratio to each other. A chemical compound has a defined chemical structure which reflects the structure at the molecular (or ionic) level. Preferably, the chemical compound is an organic compound. An “organic compound” is a chemical compound comprising carbon-hydrogen bonds (C—H bonds). Preferably, the chemical compound is an organic compound whose molecules are composed of only the following elements: Carbon (C), Hydrogen (H), Oxygen (O), Nitrogen (N), Sulfur(S), Fluorine (F), Chlorine (CI), Bromine (Br), Iodine (I) and/or Phosphorus (P).

The term “lead compound” is understood to mean a chemical compound which serves as a starting point for chemical modifications in order to generate further chemical compounds with a desired property profile.

Such a further chemical compound is also referred to in the present disclosure as a “candidate chemical compound”; this is a chemical compound for which one or more properties will be confirmed and/or determined in one or more experimental studies.

Typically, optimization of a lead structure is performed with respect to a plurality of properties that define a property profile. The properties may be physical properties, chemical properties, biological properties, and/or other properties.

Typical properties are biological activity, selectivity, toxicity, solubility, chemical stability and/or the like. Usually, each of the properties can be measured and specified by one or more values.

Prediction of one or more candidate chemical compounds is performed using a trained machine learning model.

Such a “machine learning model”, as used herein, may be understood as a computer implemented data processing architecture. The machine learning model can receive input data and provide output data based on that input data and on parameters of the machine learning model. The machine learning model can learn a relation between input data and output data through training. In training, parameters of the machine learning model may be adjusted in order to provide a desired output for a given input.

The process of training a machine learning model involves providing a machine learning algorithm (that is the learning algorithm) with training data to learn from. The term “trained machine learning model” refers to the model artifact that is created by the training process. The training data must contain the correct answer, which is referred to as the target. The learning algorithm finds patterns in the training data that map input data to the target, and it outputs a trained machine learning model that captures these patterns.

In the training process, training data are inputted into the machine learning model and the machine learning model generates an output. The output is compared with the (known) target. Parameters of the machine learning model are modified in order to reduce the deviations between the output and the (known) target to a (defined) minimum.

In general, a loss function can be used for training, where the loss function can quantify the deviations between the output and the target. The loss function may be chosen in such a way that it rewards a wanted relation between output and target and/or penalizes an unwanted relation between an output and a target. Such a relation can be, e.g., a similarity, or a dissimilarity, or another relation.

A loss function can be used to calculate a loss value for a given pair of output and target. The aim of the training process can be to modify (adjust) parameters of the machine learning model in order to reduce the loss value to a (defined) minimum. The reduction of the loss values by modifying parameters of the machine learning model can be done in an optimization procedure, such as a gradient descent procedure, for example.

A loss function may for example quantify the deviation between the output of the machine learning model for a given input and the target. If, for example, the output and the target are numbers, the loss function could be the absolute difference between these numbers. In this case, a high value of the loss function can mean that a parameter of the model needs to undergo a strong change.

In the case of vector-valued outputs, for example, difference metrics between vectors such as the root mean square error, a cosine distance, a norm of the difference vector such as a Euclidean distance, a Chebyshev distance, an Lp-norm of a difference vector, a weighted norm or any other type of difference metric of two vectors can be chosen. These two vectors may for example be the desired output (target) and the actual output.

In the case of higher dimensional outputs, such as two-dimensional, three-dimensional or higher-dimensional outputs, for example an element-wise difference metric may for example be used. Alternatively or additionally, the output data may be transformed, for example to a one-dimensional vector, before computing a loss value.

The machine learning model of the present disclosure is now described in more detail, and it is described how the machine learning model is trained to perform the prediction tasks described herein. Afterwards it is described how the trained machine learning model can be used to predict chemical candidate compounds.

The machine learning model of the present disclosure comprises an encoder-decoder structure, also referred to as autoencoder.

An autoencoder is usually used to learn efficient data encodings in an unsupervised manner. In general, the aim of an autoencoder is to learn a representation (encoding) for a set of data, typically for dimensionality reduction, by training the machine learning model to ignore “noise”. Along with the reduction side (encoder), a reconstructing side (decoder) is learnt, where the autoencoder tries to generate from the reduced encoding a representation as close as possible to its original input.

A key feature of an autoencoder is an information bottleneck between the encoder and the decoder. This bottleneck, a continuous fixed-length vector, causes the machine learning model to learn a compressed representation that captures the most statistically salient information in the data.

A multitude of vectors representing a variety of different chemical compounds span a space, also known as latent space, latent feature space or embedding space. An important feature of latent space is that it is continuous. With the help of the encoder, a molecular representation of a chemical compound consisting of discrete elements is converted into a vector in the continuous latent space in which the chemical compound is defined by numbers. The decoder can convert the vector back into a discrete molecular representation. The vector representing a chemical compound in the latent space is also referred to as “embedding” in this disclosure.

Autoencoders and their use to generate fixed-size representations of discrete molecular representations of chemical compounds in continuous latent space are well known and described in the prior art (see, e.g., R. Winter et al.: Learning continuous and data-driven molecular descriptors by translating equivalent chemical representations, Chem, Sci., 2019, 10, 1692-1701; R. Gomez-Bombarelli et al.: Automatic Chemical Design Using a Data-Driven Continuous Representation of Molecules, ACS Cent. Sci. 2018, 4, 268-276; S. Mohammadi et al.: Penalized Variational Autoencoder for Molecular Design, DOI: 10.26434/chemrxiv.7977131.v2).

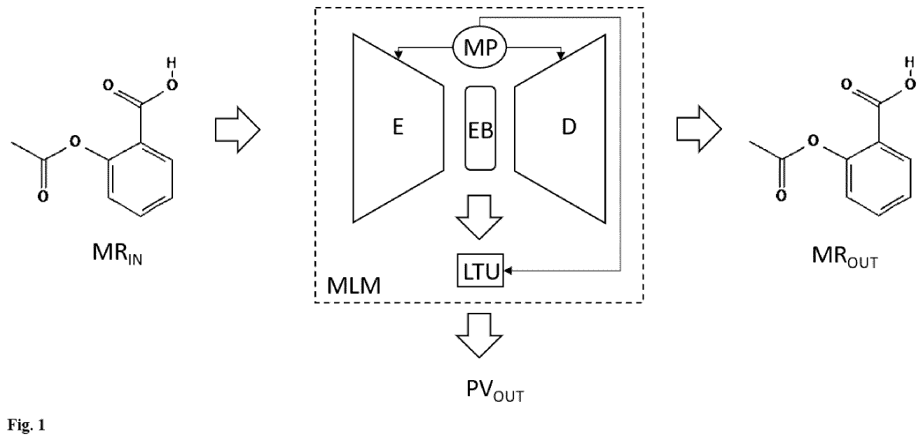

FIG. 1 shows schematically an example of a machine learning model of the present disclosure. The machine learning model MLM comprises an encoder E, a decoder D, and a linear transformation unit LTU.

The encoder E is configured to receive a molecular representation MR_{IN} of a chemical compound and to generate, at least partially on the basis of the molecular representation MR_{IN} and model parameters MP, an embedding EB representing the chemical compound in continuous latent space.

The decoder D is configured to receive an embedding EB representing a chemical compound in the continuous latent space and generate a molecular representation MR_{OUT} at least partially based on the embedding EB and model parameters MP.

The linear transformation unit LTU is configured to receive an embedding EB representing a chemical compound in the continuous latent space and to predict a property vector PV_{OUT} at least partially on the basis of the embedding EB and model parameters MP.

Preferably, a variational autoencoder is used as autoencoder, as described for example in R. Gomez-Bombarelli et al.: Automatic Chemical Design Using a Data-Driven Continuous Representation of Molecules, ACS Cent. Sci. 2018, 4, 268-276; or S. Mohammadi et al.: Penalized Variational Autoencoder for Molecular Design, DOI: 10.26434/chemrxiv.7977131.v2.

The training is performed with training data. The training data comprise, for each reference chemical compound of a multitude of reference chemical compounds, input data and target data, the input data comprising a molecular representation of the reference chemical compound, and the target data comprising a property vector representing one or more properties of the reference chemical compound.

The term “reference” is used in this disclosure to distinguish the chemical compounds used in training the machine learning model from the lead compounds and candidate chemical compounds involved in the prediction process. However, the term “reference” does not otherwise have a restrictive meaning.

The term “multitude” means more than ten, preferably more than one hundred.

It is possible to pre-train the autoencoder separately in advance. The autoencoder can be trained in an unsupervised learning procedure (see, e.g., R. Winter et al.: Learning continuous and data-driven molecular descriptors by translating equivalent chemical representations, Chem, Sci., 2019, 10, 1692-1701).

There are numerous databases that store molecular representations of reference chemical compounds that can be used to train the autoencoder as well as the whole machine learning model, such as PubChem (https://pubchem.ncbi.nlm.nih.gov). Likewise, there are several publicly available databases in which properties of reference chemical compounds are stored such as PubChem and ZINC (http://zinc.docking.org).

The molecular representation of the chemical (reference) compound can, e.g., be a SMILES, InChI, CML or WLN representation. The simplified molecular-input line-entry system (SMILES) is a specification in the form of a line notation for describing the structure of chemical species using short ASCII strings. The IUPAC International Chemical Identifier (InChI) is a textual identifier for chemical substances. Chemical Markup Language (CML) is an approach to managing molecular information using tools such as XML (Extended Markup Language) and Java. Wiswesser line notation (WLN) was one of the first line notations capable of precisely describing complex molecules.

The molecular representation of the chemical (reference) compound can also be a molecular graph. A molecular graph is a representation of the structural formula of a chemical (reference) compound in terms of graph theory. A molecular graph is a labeled graph whose vertices correspond to the atoms of the compound and edges correspond to chemical bonds. Its vertices are labeled with the kinds of the corresponding atoms and edges are labeled with the types of bonds.

The molecular representation of a chemical (reference) compound can also be the IUPAC name of the chemical (reference) compound. In chemical nomenclature, the IUPAC nomenclature of organic chemistry is a method of naming organic chemical compounds as recommended by the International Union of Pure and Applied Chemistry (IUPAC). It is published in the Nomenclature of Organic Chemistry. Ideally, every possible organic compound should have a name from which an unambiguous structural formula can be created.

Further molecular representations of chemical (reference) compounds are possible.

In a preferred embodiment, the molecular representation is a canonical SMILES code. Typically, multiple equally valid SMILES codes can be generated for a molecule. Therefore, algorithms have been developed to generate the same SMILES string for a given molecule; of the many possible strings, these algorithms select only one (see, e.g., D. Weininger et al.: SMILES. 2nd algorithm for generation of unique SMILES notation, J Chem Inf Comp Sci 1989, 29 (2): 97e101). Canonical SMILES codes are unique for each structure.

The linear transformation unit LTU serves as property prediction unit. It is configured to map an embedding in the continuous latent space to a property vector representing one or more properties. A linear transformation is a function from one vector space to another that respects the underlying (linear) structure of each vector space. In other words: a linear transformation is a mapping between two vector spaces that preserves the operations of vector addition and scalar multiplication.

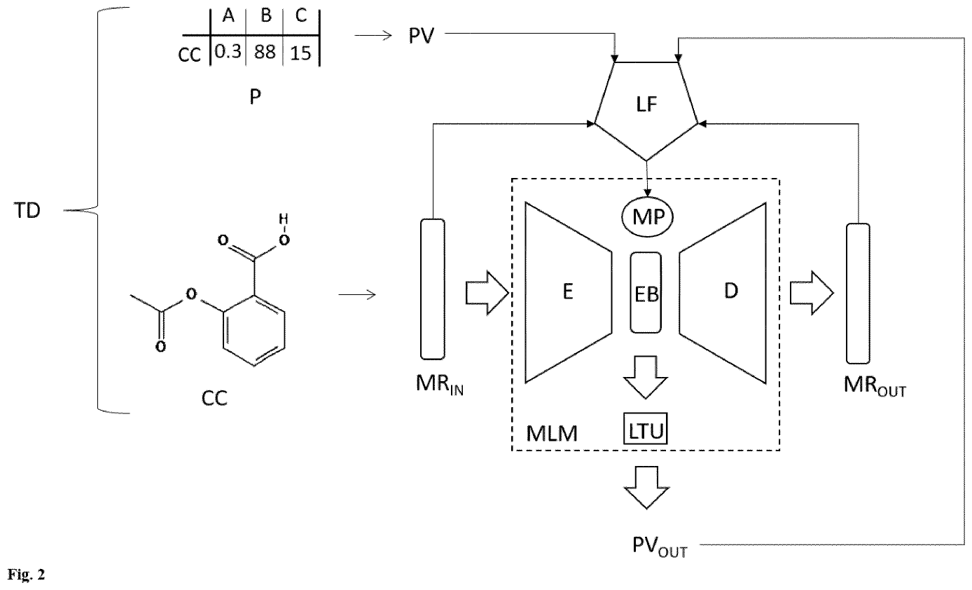

It is possible to train the machine learning model of the present disclosure to perform two tasks simultaneously: a reconstruction task and a property prediction task. Such a training is shown schematically in FIG. 2.

FIG. 2 shows the same machine learning model MLM as depicted in FIG. 1. The machine learning model MLM is trained using training data TD. The training data TD comprise, for each reference chemical compound RC of a multitude of reference chemical compounds, a molecular representation MR_{IN} and at least one property P of the reference chemical compound RC.

In FIG. 2, only one training data set comprising a molecular representation MR_{IN} of a reference chemical compound RC and property data representing the at least one property P of the reference chemical compound RC are shown. The molecular representation MR_{IN} of the reference chemical compound RC as well as the at least one property P can be inputted by a user, read from a data storage, received from another computer system and/or generated from another representation of the reference chemical compound RC. The at least one property P is usually in the form of a numerical value. In the example shown in FIG. 2, three values are present for three parameters A, B and C. Each parameter A, B and C represents one or more properties of the reference chemical compound RC. Properties of chemical compounds can be determined empirically by measurements and/or retrieved from databases. An example of a publicly available database is the ZINC database (see, e.g., https://zinc.docking.org/).

A feature vector can be generated from the at least one property of the reference chemical compound. In machine learning, a feature vector is an n-dimensional vector of numerical features that represent an object (in this case one or more properties of a chemical compound), wherein n is an integer greater than 0. The term “vector” shall also include single values, matrices, tensors, and the like. Examples of feature vector generation methods can be found in various textbooks and scientific publications (see e.g. G. A. Tsihrintzis, L. C. Jain: Machine Learning Paradigms: Advances in Deep Learning-based Technological Applications, in: Learning and Analytics in Intelligent Systems Vol. 18, Springer Nature, 2020, ISBN: 9783030497248; K. Grzegorczyk: Vector representations of text data in deep learning, Doctoral Dissertation, 2018, arXiv: 1901.01695v1 [cs.CL]; M. Ilse et al.: Attention-based Deep Multiple Instance Learning, arXiv: 1802.04712v4 [cs.LG]).

In FIG. 2, the feature vector is shown as the property vector PV. For example, it is possible that each dimension of the feature vector (property vector) represents one of the parameters A, B, and C, and the vector elements represent the values for A, B, and C, respectively.

The molecular representation MR_{IN} is fed to the encoder E as input data. The encoder E is configured and trained to generate, at least partially on the basis of the molecular representation MR_{IN} and model parameters MP, an embedding EB representing the reference chemical compound RC in continuous latent space. The decoder D is configured and trained to reconstruct, at least partially on the basis of the embedding EB and model parameters MP, the molecular representation MR_{IN}. In other words, the decoder D is configured and trained to generate and output, at least partially on the basis of the embedding EB and model parameters MP, a predicted molecular representation MR_{OUT} of the reference chemical compound which comes as close as possible to the molecular representation MR_{IN}. Simultaneously, the linear transformation unit LTU is configured and trained to predict, at least partially on the basis of the embedding EB and model parameters MP, the at least one property P. In other words, the linear transformation unit LTU is configured to generate and output a predicted property vector PV_{OUT} which comes as close as possible to the property vector PV.

The deviations between i) the target molecular representation MR_{IN} and the predicted molecular representation MR_{OUT}, and ii) the target property vector PV and the predicted property vector PV_{OUT} can be quantified using a loss function LF. Typically, the loss function LF comprises two terms, a first term that quantifies the deviations between the target molecular representation MR_{IN} and the predicted molecular representation MR_{OUT}, and a second term that quantifies the deviations between the target property vector PV and the predicted property vector PV_{OUT}. In the loss function, the two terms can be added. In the loss function, the two terms may have different weights. The weights may also vary during training.

An example of a loss function for the reconstruction task (first term) is cross-entropy loss. Examples of a loss function for the prediction task (second term) are L1 loss and/or mean squared error.

It is possible to first train the autoencoder (reconstruction task) alone (pre-training) and then train the linear transformation unit (prediction task) or then train the linear transformation unit together with the autoencoder (combined reconstruction and prediction task).

One or more loss values calculated using the loss function can be used to modify model parameters to increase the accuracy with which the machine learning model reconstructs the molecular representation and/or predicts the at least one property. For example, a high loss value may mean that one or more model parameters need to be modified to a high degree.

Usually, an optimization procedure, such as a gradient descent procedure, for example, is used to modify the model parameters in a way that leads to a reduction of loss values.

The machine learning model can be trained based on the training data until a predefined accuracy has been achieved (until the loss values have reached a pre-define minimum).

A cross-validation method can be employed to split the training data into a training dataset and a validation dataset. The training dataset can be used in the training of the machine learning model. The validation dataset can be used to verify that the results of the trained machine learning are generalizable.

The steps to train the machine learning model are summarized below:
providing and/or receiving a machine learning model, the machine learning model comprising an encoder, a decoder, and a linear transformation unit,
   wherein the encoder is configured to convert a discrete molecular representation of a chemical compound into an embedding in continuous latent space,
   wherein the decoder is configured to convert an embedding in the continuous latent space into a discrete molecular representation of a chemical compound,
   wherein the linear transformation unit is configured to map an embedding in the continuous latent space to a property vector representing one or more properties of a chemical compound,
providing and/or receiving training data, the training data comprising, for each reference chemical compound of a multitude of reference chemical compounds, input data and target data, the input data comprising a molecular representation of the reference chemical compound, the target data comprising a property vector representing one or more properties of the reference chemical compound,
training the machine learning model, wherein training comprises for each compound of the multitude of compounds:
   inputting the molecular representation of the reference chemical compound into the encoder,
   receiving a predicted molecular representation as output from the decoder,
   receiving a predicted property vector as output from the linear transformation unit,
   computing one or more loss values, the loss values quantifying deviations i) between the predicted molecular representation and the molecular representation and ii) between the predicted property vector and the property vector,
   modifying model parameters to reduce the one or more loss values,
outputting and/or storing the trained machine learning model and/or the modified model parameters.

Once the machine learning model is trained, it can be used for prediction purposes.

According to the present disclosure, the trained machine learning model is used to propose (predict) one or more candidate chemical compounds with a desired property profile based on at least two lead compounds.

Each lead compound is characterized by a molecular representation and by at least one property. The at least one candidate chemical compound is a chemical compound that is structurally and in terms of its property(ies) intermediate between the two lead compounds. Thus, the at least one candidate chemical compound has a chemical structure that is similar to the first lead compound and the second lead compound. The at least one candidate chemical compound is obtained by interpolation within the continuous latent space using the trained machine learning model.

A first embedding representing the first lead compound in the continuous latent space may be generated for the first lead compound based on the molecular representation of the first lead compound using the encoder of the trained machine learning model. Similarly, a second embedding representing the second lead compound in continuous latent space may be generated for the second lead compound based on the molecular representation of the second lead compound using the encoder of the trained machine learning model.

Moving within the continuous latent space from the embedding of the first lead compound to the embedding of the second lead compound, each point within the continuous latent space corresponds to an embedding of a further chemical compound (it should be noted, however, that a valid chemical structure cannot be generated for every point of the continuous latent space using the decoder; more on this is given later in the description).

The closer the point is to the embedding of the first lead compound, the more similar the chemical structure of the further chemical compound is to the chemical structure of the first lead compound; the closer the point is to the embedding of the second lead compound, the more similar the chemical structure of the further chemical compound is to the chemical structure of the second lead compound.

This applies analogously to the at least one property: the closer the point is to the embedding of the first lead compound, the more similar the at least one property of the further chemical compound is to the least one property of the first lead compound; the closer the point is to the embedding of the second lead compound, the more similar the least one property of the further chemical compound is to the least one property of the second lead compound.

If one moves within the continuous latent space along a straight line from the embedding of the first lead compound to the embedding of the second lead compound and converts the embeddings lying on the straight line into molecular representations with the aid of the decoder, the chemical structure of the first lead compound is gradually changed in the direction of the second lead compound. A morphing of the chemical structure of the first lead compound into the chemical structure of the second lead compound takes place.

At the same time, the at least one property changes from the at least one property of the first lead compound to the at least one property of the second lead compound. Thus, morphing of the at least one property also takes place.

In other words, one can identify within the continuous latent space, by interpolation between the embedding of the first lead compound and the embedding of the second lead compound, one or more further embeddings belonging to chemical compounds that are structurally and with respect to the at least one property intermediate between the lead compounds.

The fact that both the chemical structures and the properties of the chemical compounds in the continuous latent space change gradually from one lead compound to the other lead compound, and that one can identify new chemical compounds that lie between the lead compounds in terms of their chemical structures and properties by interpolation, is because the machine learning model has been trained to both reconstruct molecular representations and predict a property vectors, and because the property vectors result from embeddings by a linear transformation.

Thus, by choosing the first lead compound and choosing the second lead compound, one can choose which chemical structure the at least one candidate chemical compound should be based on and which property(ies) it should possess. For example, if one has two lead compounds that are structurally very different but similar with respect to their at least one property, one can identify by interpolation in the continuous latent space one or more chemical candidate compounds that are structurally intermediate between the lead compounds but in which the at least one property is retained.

If one has a first lead compound that is difficult to synthesize but has a desired high value of a property (e.g., biological activity), and a second lead compound that is easy to synthesize but has a lower property value, one can identify, by interpolation within the continuous latent space between the embeddings of the two lead compounds, one or more embeddings of candidate chemical compounds that may represent a trade-off between ease of synthesis and property value.

If one lead compound has one desired property and another lead compound has another desired property, a third lead compound can be identified that has both desired properties at least in part.

The synthetic chemist can think of numerous other ways in which the teachings of the present disclosure can be used to efficiently identify new candidate chemical compounds.

The steps for predicting candidate chemical compounds are described in more detail below.

In a first step, at least two lead compounds, a first lead compound and a second lead compound are specified and/or selected. The lead compounds can be, e.g., specified and/or selected by a user. The lead compounds can be entered into the computer system of the present disclosure by the user, or they can be stored in a data memory and read out from the data memory by the computer system of the present disclosure.

If a molecular representation of each of the lead compounds is not yet available, it can be generated from another representation according to the usual procedures described in the prior art, including manual conversion by a chemist.

The at least two lead compounds may be selected based on at least one property, e.g., at least two lead compounds may be selected that have one or more desired properties. In other words: the one or more properties of one or more lead compounds can serve as one or more target property(ies). The molecular representation of the first lead compound is inputted into the trained machine learning model. More precisely, the molecular representation of the first lead compound is fed to the encoder of the trained machine learning model. The encoder is configured and trained to convert the molecular representation of the first lead compound into a first embedding. The first embedding is a representation of the first lead compound in the continuous latent space.

Since the machine learning model was trained not only to reconstruct molecular representations but also to predict at least one property based on the molecular representation, the first embedding represents not only the molecular representation of the first lead compound but also the at least one property of the first lead compound in the continuous latent space.

The molecular representation of the second lead compound is also inputted into the trained machine learning model. More precisely, the molecular representation of the second lead compound is fed to the encoder of the trained machine learning model. The encoder is configured and trained to convert the molecular representation of the second lead compound into a second embedding. The second embedding is a representation of the second lead compound in the continuous latent space. The second embedding represents not only the molecular representation of the second lead compound but also the at least one property of the second lead compound in the continuous latent space.

In a further step a point within the continuous latent space that lies between the first embedding and the second embedding is selected. The point defines a third embedding. The third embedding is a representation of a further chemical compound. The further chemical compound is also referred to as “candidate chemical compound” in this disclosure. The third embedding represents a molecular representation as well as at least one property of the further chemical compound in the continuous latent space. Multiple third embeddings can also be selected, such as two or three or four or more.

The selection can be made by moving from the first embedding towards the second embedding or vice versa. The movement can be along a straight line connecting the first embedding and the second embedding. Along the straight line, one or more points can be selected that define one or more third embeddings.

The closer a selected embedding is to the first embedding, the more similar the chemical structure and the at least one property of the respective chemical compound represented by the selected embedding is to the chemical structure and the at least one property of the first lead compound. The closer a selected embedding is to the second embedding, the more similar the chemical structure and the at least one property of the respective chemical compound represented by the selected embedding is to the chemical structure and the at least one property of the second lead compound.

It is also possible to specify and/or select three lead compounds, a first lead compound, a second lead compound and a third lead compound. Three embeddings can be generated from the molecular representation of each of the three lead compounds using the encoder: a first embedding representing the first lead compound in the continuous latent space, a second embedding representing the second lead compound in the continuous latent space, and a third embedding representing the third lead compound in the continuous latent space. One or more fourth embeddings within the continuous latent space can be selected that lie between the first embedding, second embedding, and third embedding. The one or more fourth embeddings represent the chemical structure and at least one property of one or more further chemical compounds. The chemical structure and the at least one property of a further chemical compound are between the chemical structure and the property of the first, second and third lead compounds.

This procedure can be applied to any number of lead structures.

In a preferred embodiment, a number n of embeddings representing a number n of lead compounds in the continuous latent space are generated using the encoder, and an embedding within the continuous latent space is identified that lies, e.g., at the centroid (geometric center) of the n embeddings, wherein n is a number greater than 1.

Once one or more embeddings that lie within the continuous latent space between the embeddings of two or more lead compounds have been identified, the associated molecular representations and/or properties can be determined.

A molecular representation of an embedding can be generated by feeding the embedding to the decoder of the trained machine learning model. The decoder is configured and trained to convert the embedding into the molecular representation. The molecular representation can be outputted, e.g., displayed on a computer display, printed via a printer and/or stored on a data memory.

A property vector representing the at least one property can be generated by feeding the embedding to the linear transformation unit of the trained machine learning model. The linear transformation unit is configured to map the embedding to the property vector. The at least one property obtained from the property vector can be outputted, e.g., displayed on a computer display, printed via a printer and/or stored on a data memory.

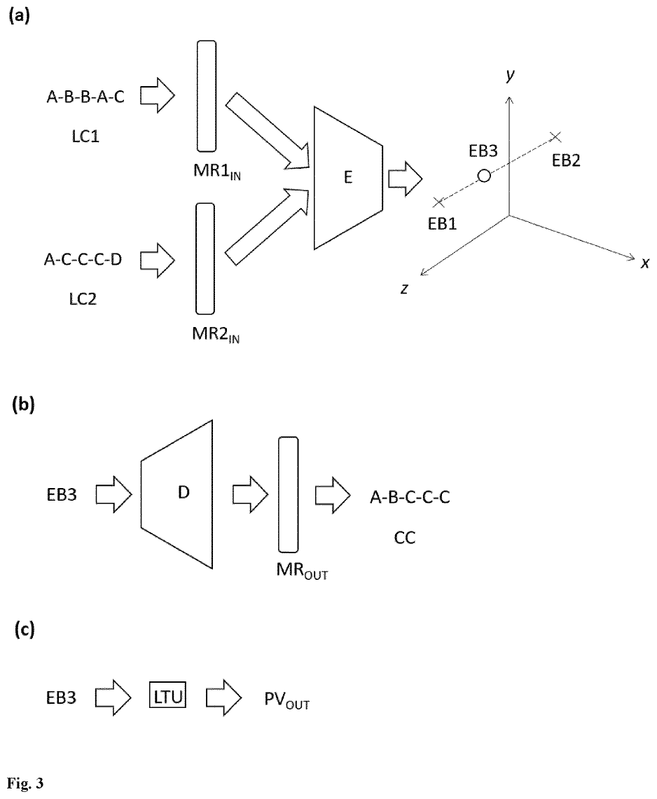

FIG. 3 shows schematically by way of example the use of the trained machine learning model for prediction.

In FIG. 3 (a), a first molecular representation MR1_{IN} of a first lead compound LC1 and a second molecular representation MR2_{IN} of a second lead compound LC2 are generated. The molecular representations MR1_{IN} and MR2_{IN} are fed to the encoder E of the trained machine learning model. The encoder E generates a first embedding EB1 representing the first lead compound LC1 in continuous latent space, and the encoder E generates a second embedding EB2 representing the second lead compound LC2 in continuous latent space. The continuous latent space is represented in FIG. 3 (a) by a Cartesian coordinate system with dimensions x, y, and z.

A third embedding EB3 is selected. The third embedding EB3 lies within the continuous latent space between the first embedding EP1 and the second embedding EP2. The third embedding represents a candidate chemical compound in the continuous latent space.

In FIG. 3 (b) the third embedding EB3 is fed to the decoder D of the trained machine learning model. The decoder D generates a molecular representation MR_{OUT}. The molecular representation MR_{OUT} is a molecular representation of the candidate chemical compound CC. The chemical structure of the candidate chemical compound CC lies between the chemical structures of the lead compounds LC1 and LC2.

In FIG. 3 (c) the third embedding EB3 is fed to the linear transformation unit LTU of the trained machine learning model. The linear transformation unit LTU generates a property vector PV_{OUT}. The property vector PV_{OUT} represents one or more properties of the candidate chemical compound CC. The value(s) of the one or more properties of the candidate chemical compound CC lie(s) between the values of the one or more or properties of the lead compounds LC1 and LC2.

In a further step, it can be checked whether a molecular representation generated by the decoder of the trained machine learning model is a valid molecular representation, i.e., a representation of a chemical structure of a chemical compound that can actually exist, i.e., can be synthesized.

If the molecular representation is a SMILES code, this SMILES code can be validated, for example, using the freely available open-source cheminformatics software RDKit (see, e.g., http://www.rdkit.org).

Invalid molecular representations may be discarded. In case of an invalid molecular representation, another embedding between the embeddings representing lead compounds in the continuous latent space can be selected and the respective molecular representation can be generated using the decoder of the trained machine learning model.

It is possible that the chemical compound with the molecular representation generated by the decoder has one or more properties that deviate from the desired property profile. Such deviations can arise, for example, because jumps can occur during the conversion from an embedding in the continuous latent space to a discrete structure, resulting in altered properties.

In a further step, it can be checked whether the property profile of the candidate chemical compound matches the target property profile. The target property profile can be one or more properties of one or more lead compounds and/or a mean value (e.g., an arithmetic mean value) of one or more properties of one or more lead compounds.

The (optionally validated) molecular representation of the chemical compound can be inputted into the encoder of the trained machine learning model. The encoder generates an embedding representing the candidate chemical compound in the continuous latent space. The embedding representing the candidate chemical compound in the continuous latent space is then inputted into the linear transformation unit in order to predict the at least one property of the candidate chemical compound. The linear transformation unit outputs a property vector representing the one or more properties of the candidate chemical compound. The one or more properties can be compared with the target property profile. The comparison can be based on property vectors; one property vector can represent one or more desired properties (target property vector), the other property vector can represent one or more predicted properties of the candidate chemical compound.

In such a comparison, a measure of similarity of the two vectors can be determined. A measure of the similarity of two vectors can be, for example, a similarity value, such as the cosine similarity, or a distance measure, such as the Euclidean distance, the Manhattan distance, Chebyshev distance, Minkowski distance, weighted Minkowski distance, Mahalanobis distance, Hamming distance, Canberra distance, Bray Curtis distance, or a combination thereof.

A distance d(TPV, PV_{TC}) between a target property vector TPV and a predicted property vector PV_{TC} representing one or more properties of a candidate chemical compound can be converted into a similarity value s(TPV, PV_{TC}), e.g., by the following equation:

$$s ( TPV , {PV}_{TC} ) = \frac{1}{1 + d ( TPV , {PV}_{TC} )}$$

The similarity value (or the distance value or any other measure of the similarity of the two vectors TPV and PV_{TC}) can be compared with a pre-defined threshold. This is explained by the example of a similarity value which is always positive and takes the value 1 (or 100%) if two vectors are identical and takes the value 0 if two vectors have no similarity. For example, the pre-defined threshold may be 0.8 or 0.85 or 0.9 or 0.91 or 0.95 or 0.99 or some other value.

If the similarity value is smaller than the pre-defined threshold, it may mean that the property profile of the candidate chemical compound is so far away from the target property profile that the candidate chemical compound is not a promising candidate for further investigation and can be discarded. In such a case, another embedding between the embeddings representing lead compounds in the continuous latent space can be selected.

In case a promising candidate chemical compound has been identified, it can be synthesized and/or characterized, i.e., one or more properties can be measured on the candidate chemical compound.

Synthesizing a chemical compound and measuring its properties is not normally a step of the process of the invention, particularly since the process of the invention is a computer-implemented invention. However, it is possible that an examiner of a patent office may require that the process according to the invention have a direct effect on the real physical world beyond the usual interaction between hardware and software in running the computer-implemented process on a computer system.

Steps that may be part of the computer-implemented method may be steps to initiate synthesis of the candidate chemical compound (e.g., sending a message to a synthesis laboratory as to which candidate chemical compound to synthesize and/or automatically ordering chemicals to produce the candidate chemical compound) and/or steps to initiate measurement of one or more properties of the candidate chemical compound (e.g., sending a message to a measurement laboratory as to which property to measure on which candidate chemical compound).

Steps that may be performed following the computer-implemented method include synthesizing the compound and/or performing a measurement of one or more properties on the candidate chemical compound (e.g., to verify the predicted property(ies)).

The approach described herein effectively and efficiently leads to new candidates for lead structure optimization.

The operations in accordance with the teachings herein may be performed by at least one computer system specially constructed for the desired purposes or general-purpose computer system specially configured for the desired purpose by at least one computer program stored in a typically non-transitory computer readable storage medium.

A “computer system” is a system for electronic data processing that processes data by means of programmable calculation rules. Such a system usually comprises a “computer”, that unit which comprises a processor for carrying out logical operations, and also peripherals.

In computer technology, “peripherals” refer to all devices which are connected to the computer and serve for the control of the computer and/or as input and output devices. Examples thereof are monitor (screen), printer, scanner, mouse, keyboard, drives, camera, microphone, loudspeaker, etc. Internal ports and expansion cards are, too, considered to be peripherals in computer technology.

Computer systems of today are frequently divided into desktop PCs, portable PCs, laptops, notebooks, netbooks and tablet PCs and so-called handhelds (e.g. smartphone); all these systems can be utilized for carrying out the invention.

The term “non-transitory” is used herein to exclude transitory, propagating signals or waves, but to otherwise include any volatile or non-volatile computer memory technology suitable to the application.

The term “computer” should be broadly construed to cover any kind of electronic device with data processing capabilities, including, by way of non-limiting example, personal computers, servers, embedded cores, computing system, communication devices, processors (e.g., digital signal processor (DSP)), microcontrollers, field programmable gate array (FPGA), application specific integrated circuit (ASIC), etc.) and other electronic computing devices.

The term “process” as used above is intended to include any type of computation or manipulation or transformation of data represented as physical, e.g., electronic, phenomena which may occur or reside e.g., within registers and/or memories of at least one computer or processor. The term processor includes a single processing unit or a plurality of distributed or remote such units.

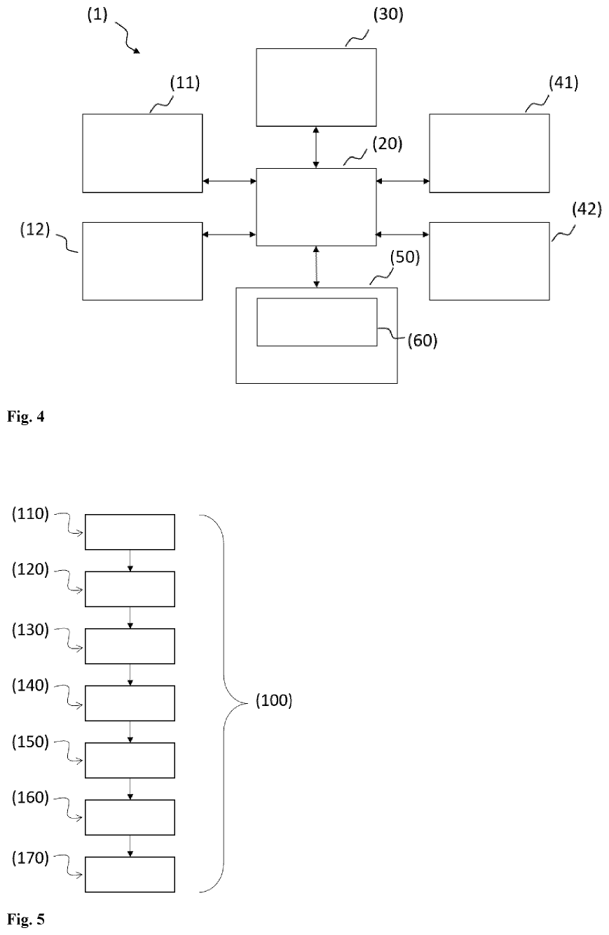

FIG. 4 illustrates a computer system (1) according to some example implementations of the present disclosure in more detail. The computer may include one or more of each of a number of components such as, for example, processing unit (20) connected to a memory (50) (e.g., storage device).

The processing unit (20) may be composed of one or more processors alone or in combination with one or more memories. The processing unit is generally any piece of computer hardware that is capable of processing information such as, for example, data, computer programs and/or other suitable electronic information. The processing unit is composed of a collection of electronic circuits some of which may be packaged as an integrated circuit or multiple interconnected integrated circuits (an integrated circuit at times more commonly referred to as a “chip”). The processing unit may be configured to execute computer programs, which may be stored onboard the processing unit or otherwise stored in the memory (50) of the same or another computer.

The processing unit (20) may be a number of processors, a multi-core processor or some other type of processor, depending on the particular implementation. Further, the processing unit may be implemented using a number of heterogeneous processor systems in which a main processor is present with one or more secondary processors on a single chip. As another illustrative example, the processing unit may be a symmetric multi-processor system containing multiple processors of the same type. In yet another example, the processing unit may be embodied as or otherwise include one or more ASICs, FPGAs or the like. Thus, although the processing unit may be capable of executing a computer program to perform one or more functions, the processing unit of various examples may be capable of performing one or more functions without the aid of a computer program. In either instance, the processing unit may be appropriately programmed to perform functions or operations according to example implementations of the present disclosure.

The memory (50) is generally any piece of computer hardware that is capable of storing information such as, for example, data, computer programs (e.g., computer-readable program code (60)) and/or other suitable information either on a temporary basis and/or a permanent basis. The memory may include volatile and/or non-volatile memory, and may be fixed or removable. Examples of suitable memory include random access memory (RAM), read-only memory (ROM), a hard drive, a flash memory, a thumb drive, a removable computer diskette, an optical disk, a magnetic tape or some combination of the above. Optical disks may include compact disk-read only memory (CD-ROM), compact disk-read/write (CD-R/W), DVD, Blu-ray disk or the like. In various instances, the memory may be referred to as a computer-readable storage medium. The computer-readable storage medium is a non-transitory device capable of storing information, and is distinguishable from computer-readable transmission media such as electronic transitory signals capable of carrying information from one location to another. Computer-readable medium as described herein may generally refer to a computer-readable storage medium or computer-readable transmission medium.

In addition to the memory (50), the processing unit (20) may also be connected to one or more interfaces for displaying, transmitting and/or receiving information. The interfaces may include one or more communications interfaces and/or one or more user interfaces. The communications interface(s) may be configured to transmit and/or receive information, such as to and/or from other computer(s), network(s), database(s) or the like. The communications interface may be configured to transmit and/or receive information by physical (wired) and/or wireless communications links. The communications interface(s) may include interface(s) (41) to connect to a network, such as using technologies such as cellular telephone, Wi-Fi, satellite, cable, digital subscriber line (DSL), fiber optics and the like. In some examples, the communications interface(s) may include one or more short-range communications interfaces (42) configured to connect devices using short-range communications technologies such as NFC, RFID, Bluetooth, Bluetooth LE, ZigBee, infrared (e.g., IrDA) or the like.

The user interfaces may include a display (30). The display may be configured to present or otherwise display information to a user, suitable examples of which include a liquid crystal display (LCD), light-emitting diode display (LED), plasma display panel (PDP) or the like. The user input interface(s) (11) may be wired or wireless, and may be configured to receive information from a user into the computer system (1), such as for processing, storage and/or display. Suitable examples of user input interfaces include a microphone, image or video capture device, keyboard or keypad, joystick, touch-sensitive surface (separate from or integrated into a touchscreen) or the like. In some examples, the user interfaces may include automatic identification and data capture (AIDC) technology (12) for machine-readable information. This may include barcode, radio frequency identification (RFID), magnetic stripes, optical character recognition (OCR), integrated circuit card (ICC), and the like. The user interfaces may further include one or more interfaces for communicating with peripherals such as printers and the like.

As indicated above, program code instructions may be stored in memory, and executed by processing unit that is thereby programmed, to implement functions of the systems, subsystems, tools and their respective elements described herein. As will be appreciated, any suitable program code instructions may be loaded onto a computer or other programmable apparatus from a computer-readable storage medium to produce a particular machine, such that the particular machine becomes a means for implementing the functions specified herein. These program code instructions may also be stored in a computer-readable storage medium that can direct a computer, processing unit or other programmable apparatus to function in a particular manner to thereby generate a particular machine or particular article of manufacture. The instructions stored in the computer-readable storage medium may produce an article of manufacture, where the article of manufacture becomes a means for implementing functions described herein. The program code instructions may be retrieved from a computer-readable storage medium and loaded into a computer, processing unit or other programmable apparatus to configure the computer, processing unit or other programmable apparatus to execute operations to be performed on or by the computer, processing unit or other programmable apparatus.

Retrieval, loading and execution of the program code instructions may be performed sequentially such that one instruction is retrieved, loaded and executed at a time. In some example implementations, retrieval, loading and/or execution may be performed in parallel such that multiple instructions are retrieved, loaded, and/or executed together. Execution of the program code instructions may produce a computer-implemented process such that the instructions executed by the computer, processing circuitry or other programmable apparatus provide operations for implementing functions described herein.

Execution of instructions by processing unit, or storage of instructions in a computer-readable storage medium, supports combinations of operations for performing the specified functions. In this manner, a computer system (1) may include processing unit (20) and a computer-readable storage medium or memory (50) coupled to the processing circuitry, where the processing circuitry is configured to execute computer-readable program code (60) stored in the memory. It will also be understood that one or more functions, and combinations of functions, may be implemented by special purpose hardware-based computer systems and/or processing circuitry which perform the specified functions, or combinations of special purpose hardware and program code instructions.

FIG. 5 shows an embodiment of the method for identifying a candidate chemical compound having a desired property profile in form of a flow chart. The method (100) comprises the steps:
(110) providing a trained machine learning model, the trained machine learning model comprising an encoder, a decoder, and a linear transformation unit,
   wherein the encoder is configured and trained to convert a discrete molecular representation of a chemical compound into an embedding in continuous latent space,
   wherein the decoder is configured and trained to convert an embedding in the continuous latent space into a discrete molecular representation of a chemical compound,
   wherein the linear transformation unit is configured and trained to map an embedding in the continuous latent space to a property vector representing one or more properties of a chemical compound,
(120) receiving a first molecular representation of a first lead compound and a second molecular representation of a second lead compound,
(130) converting the first molecular representation of the first lead compound into a first embedding representing the first lead compound in the continuous latent space via the encoder,
(140) converting the second molecular representation of the second lead compound into a second embedding representing the second lead compound in the continuous latent space via the encoder,
(150) selecting a third embedding within the continuous latent space between the first embedding and the second embedding, the third embedding representing the candidate chemical compound in the continuous latent space,
(160) converting the third embedding into a discrete molecular representation of the candidate chemical compound via the decoder,
(170) outputting the discrete molecular representation of the candidate chemical compound and/or another representation of the candidate chemical compound.

1. A method for identifying chemical compounds, the method comprising:
providing a trained machine learning model (MLM), the trained machine learning model (MLM) comprising an encoder (E), a decoder (D), and a linear transformation unit (LTU),
wherein the encoder (E) is configured and trained to convert a discrete molecular representation (MR_{IN}) of a chemical compound (RC) into an embedding (EB) in continuous latent space,
wherein the decoder (D) is configured and trained to convert an embedding (EB) in the continuous latent space into a discrete molecular representation (MR_{IN}) of a chemical compound (RC),
wherein the linear transformation unit (LTU) is configured and trained to map an embedding (EB) in the continuous latent space to a property vector (PV_{OUT}) representing one or more properties of a chemical compound (RC);
receiving a first molecular representation (MR1_{IN}) of a first lead compound (LC1) and a second molecular representation (MR2_{IN}) of a second lead compound (LC2);
converting the first molecular representation (MR1_{IN}) of the first lead compound (LC1) into a first embedding (EB1) representing the first lead compound (LC1) in the continuous latent space via the encoder (E);
converting the second molecular representation (MR2_{IN}) of the second lead compound (LC2) into a second embedding (EB2) representing the second lead compound (LC2) in the continuous latent space via the encoder (E);
selecting a third embedding (EB3) within the continuous latent space between the first embedding (EB1) and the second embedding (EB2), the third embedding (EB3) representing a candidate chemical compound (CC) in the continuous latent space;
converting the third embedding (EB3) into a discrete molecular representation (MR_{OUT}) of the candidate chemical compound (CC) via the decoder (D); and
outputting the discrete molecular representation (MR_{OUT}) of the candidate chemical compound (CC) and/or another representation of the candidate chemical compound (CC).

2. The method according to claim 1, further comprising:
mapping the third embedding (EB3) to a property vector (PV_{OUT}) representing one or more properties of the candidate chemical compound (CC) via the linear transformation unit (LTU); and
outputting the one or more properties of the candidate chemical compound (CC).

3. The method according to claim 1, further comprising:
checking whether the molecular representation (MR_{OUT}) of the candidate chemical compound (CC) is a valid molecular representation; and
if the molecular representation (MR_{OUT}) of the candidate chemical compound (CC) is not a valid molecular representation:
discarding the candidate chemical compound (CC);
selecting a further third embedding within the continuous latent space between the first embedding (EB1) and the second embedding (EB2);
converting the further third embedding into a discrete molecular representation of a further candidate chemical compound; and
outputting the discrete molecular representation of the further candidate chemical compound and/or another representation of the further candidate chemical compound.

4. The method according to claim 1, further comprising:
providing and/or receiving a target property vector, the target property vector representing one or more desired properties of the candidate chemical compound (CC);
converting the molecular representation (MR_{OUT}) of the candidate chemical compound (CC) into an embedding representing the candidate chemical compound (CC) in the continuous latent space via the encoder (E);
mapping the embedding representing the candidate chemical compound (CC) to a predicted property vector representing one or more properties of the candidate chemical compound (CC) via the linear transformation unit (LTU);
comparing the target property vector with the predicted property vector; and
if the deviation between the target property vector and the predicted property vector exceeds a defined threshold:
discarding the candidate chemical compound (CC);
selecting a further third embedding within the continuous latent space between the first embedding (EB1) and the second embedding (EB);
converting the further third embedding into a discrete molecular representation of a further candidate chemical compound; and
outputting the discrete molecular representation of the further candidate chemical compound and/or another representation of the further candidate chemical compound.

5. The method according to claim 1, wherein the one or more properties are one of more of the following properties: biological activity, selectivity, toxicity, solubility, chemical stability.

6. The method according to claim 1, wherein the molecular representations (MR1_{IN}, MR2_{IN}) of lead compounds (LC1, LC2) are SMILES codes preferably canonical SMILES codes.

7. The method according to claim 1, wherein the linear transformation unit (LTU) is configured to linearly transform the third embedding (EB3) into the property vector (PV_{OUT}).

8. The method according to claim 1, further comprising:
receiving a number n of molecular representations (MR1_{IN}, MR2_{IN}) of a number n of lead compounds (LC1, LC2), wherein n is an integer greater than one;
converting the molecular representation (MR1_{IN}, MR2_{IN}) of each lead compound (LC1, LC2) into an embedding (EB1, EB2) representing the lead compound (LC1, LC2) in the continuous latent space via the encoder (E);
selecting a further embedding (EB3) within the continuous latent space between the embeddings (EB1, EB2) of the lead compounds (LC1, LC2);
converting the further embedding (EB3) into a discrete molecular representation (MR_{OUT}) of a candidate chemical compound (CC); and
outputting the discrete molecular representation (MR_{OUT}) of the candidate chemical compound (CC) and/or another representation of the candidate chemical compound (CC).

9. The method according to claim 8, wherein the further embedding lies at a centroid of the embeddings (EB1, EB2) of the lead compounds (LC1, LC2).

10. The method according to claim 1, further comprising:
initiating synthesis and/or characterization of the candidate chemical compound (CC).

11. The method according to claim 1,
wherein the method is a computer-implemented method.

12. The method according to claim 1, further comprising:
synthesizing the candidate chemical compound (CC) and/or measuring one or more properties on the candidate chemical compound (CC).

13. The method according to claim 1, wherein training of the machine learning model (MLM) comprises:
providing and/or receiving a machine learning model (MLM), the machine learning model (MLM) comprising an encoder (E), a decoder (D), and a linear transformation unit (LTU),
wherein the encoder (E) is configured to convert a discrete molecular representation (MR_{IN}) of a chemical compound (RC) into an embedding (EB) in continuous latent space,
wherein the decoder (D) is configured to convert an embedding (EB) in the continuous latent space into a discrete molecular representation (MR_{OUT}) of a chemical compound (RC),
wherein the linear transformation unit (LTU) is configured to map an embedding (EB) in the continuous latent space to a property vector (PV_{OUT}) representing one or more properties (P) of a chemical compound;
providing and/or receiving training data (TD), the training data comprising, for each reference chemical compound (RC) of a multitude of reference chemical compounds, input data and target data, the input data comprising a molecular representation (MR_{IN}) of the reference chemical compound (RC), the target data comprising a property vector (PV) representing one or more properties (P) of the reference chemical compound (RC);
training the machine learning model (MLM), wherein training comprises for each reference chemical compound of the multitude of reference chemical compounds:
inputting the molecular representation (MR_{IN}) of the reference chemical compound (RC) into the encoder (E);
receiving a predicted molecular representation (MR_{OUT}) as output from the decoder (D);
receiving a predicted property vector (PV_{OUT}) as output from the linear transformation unit (LTU);
computing one or more loss values by means of a loss function (LF), the loss values quantifying deviations i) between the predicted molecular representation (MR_{OUT}) and the molecular representation (MR_{IN}) and ii) between the predicted property vector (PV_{OUT}) and the property vector (PV); and
modifying model parameters (MP) to reduce the one or more loss values; and
outputting and/or storing the trained machine learning model (ML) and/or the modified model parameters (MP).

14. A computer system comprising:
a processor; and
a memory storing an application program configured to perform, when executed by the processor, an operation, the operation comprising:
providing a trained machine learning model (MLM), the trained machine learning model (MLM) comprising an encoder (E), a decoder (D), and a linear transformation unit (LTU),
wherein the encoder (E) is configured and trained to convert a discrete molecular representation (MR_{IN}) of a chemical compound (RC) into an embedding (EB) in continuous latent space,
wherein the decoder (D) is configured and trained to convert an embedding (EB) in the continuous latent space into a discrete molecular representation (MR_{IN}) of a chemical compound (RC),
wherein the linear transformation unit (LTU) is configured and trained to map an embedding (EB) in the continuous latent space to a property vector (PV_{OUT}) representing one or more properties of a chemical compound (RC);
receiving a first molecular representation (MR1_{IN}) of a first lead compound (LC1) and a second molecular representation (MR2_{IN}) of a second lead compound (LC2);
converting the first molecular representation (MR1_{IN}) of the first lead compound (LC1) into a first embedding (EB1) representing the first lead compound (LC1) in the continuous latent space via the encoder (E);
converting the second molecular representation (MR2_{IN}) of the second lead compound (LC2) into a second embedding (EB2) representing the second lead compound (LC2) in the continuous latent space via the encoder (E);
selecting a third embedding (EB3) within the continuous latent space between the first embedding (EB1) and the second embedding (EB2), the third embedding (EB3) representing a candidate chemical compound (CC) in the continuous latent space;
converting the third embedding (EB3) into a discrete molecular representation (MR_{OUT}) of the candidate chemical compound (CC) via the decoder (D); and
outputting the discrete molecular representation (MR_{OUT}) of the candidate chemical compound (CC) and/or another representation of the candidate chemical compound (CC).

15. A non-transitory computer readable medium having stored thereon software instructions that, when executed by a processor of a computer system, cause the computer system to execute the following steps:
providing a trained machine learning model (MLM), the trained machine learning model (MLM) comprising an encoder (E), a decoder (D), and a linear transformation unit (LTU),
wherein the encoder (E) is configured and trained to convert a discrete molecular representation (MR_{IN}) of a chemical compound (RC) into an embedding (EB) in continuous latent space,
wherein the decoder (D) is configured and trained to convert an embedding (EB) in the continuous latent space into a discrete molecular representation (MR_{IN}) of a chemical compound (RC),
wherein the linear transformation unit (LTU) is configured and trained to map an embedding (EB) in the continuous latent space to a property vector (PV_{OUT}) representing one or more properties of a chemical compound (RC);
receiving a first molecular representation (MR1_{IN}) of a first lead compound (LC1) and a second molecular representation (MR2_{IN}) of a second lead compound (LC2);
converting the first molecular representation (MR1_{IN}) of the first lead compound (LC1) into a first embedding (EB1) representing the first lead compound (LC1) in the continuous latent space via the encoder (E);
converting the second molecular representation (MR2_{IN}) of the second lead compound (LC2) into a second embedding (EB2) representing the second lead compound (LC2) in the continuous latent space via the encoder (E);
selecting a third embedding (EB3) within the continuous latent space between the first embedding (EB1) and the second embedding (EB2), the third embedding (EB3) representing a candidate chemical compound (CC) in the continuous latent space;
converting the third embedding (EB3) into a discrete molecular representation (MR_{OUT}) of the candidate chemical compound (CC) via the decoder (D); and
outputting the discrete molecular representation (MR_{OUT}) of the candidate chemical compound (CC) and/or another representation of the candidate chemical compound (CC).

16. The method according to claim 6, wherein the molecular representations (MR1_{IN}, MR2_{IN}) of lead compounds (LC1, LC2) are canonical SMILES codes.
